(19) United States
(12) Patent Application Publication
(10) Pub. No.: US 2009/0131500 A1
(43) Pub. Date: May 21, 2009

(54) PORPHYRIN DERIVATES AND THEIR USE AS PHOTOSENSITIZERS IN PHOTODYNAMIC THERAPY

(76) Inventors: Beate Roeder, Falkensee (DE); Eugeny Ermilov, Berlin (DE); Norbert Jux, Langensendelbach (DE); Stefan Jasinski, Igensdorf (DE)

Correspondence Address: ROTHWELL, FIGG, ERNST & MANBECK, P.C., 1425 K STREET, N.W., SUITE 800, WASHINGTON, DC, 20005, US

(21) Appl. No.: 12/282,436

(22) PCT Filed: Mar. 9, 2007
(86) PCT No.: PCT/EP07/52251

§ 371 Date: Sep. 10, 2008

(30) Foreign Application Priority Data
Mar. 10, 2006 (EP) 06075595.6

(51) Int. Cl. A61K 31/409; C07D 487/04; A61P 35/00

(52) U.S. Cl. 514/410; 540/476

(57) ABSTRACT

The present invention relates to novel porphyrin derivates of formula (I), especially to porphyrins which carry substituted phenyl moieties in meso-position. The porphyrin derivates of the invention are useful as photosensitizers in photodynamic therapy (PDT), especially in photodynamic tumor therapy. Due to their excellent photophysical properties the compounds of formula I are extremely efficient in photodynamic treatment.

The present invention relates to novel porphyrin derivates of formula I, especially to porphyrins with four substituted phenyl moieties in meso-position. The porphyrin derivatives of the invention are useful as photosensitizers in photodynamic therapy (PDT), especially in photodynamic tumor therapy. Due to their excellent photophysical properties the compounds of formula I are extremely efficient in photodynamic treatment.

Photodynamic treatment is a procedure that uses photoselective (light-activated) compounds to target and destroy diseased cells. The photoselective compounds which are also called photosensitizers are inactive until switched on by light of a specific wavelength. By absorbing the light, energy is transferred from the photosensitizer to molecular oxygen generating singlet molecular oxygen (¹O₂) which plays the key role in PDT and causes direct or indirect photodamage of the target tissue.

A large number of naturally occurring and synthetic dyes have been evaluated as potential photosensitizers in photodynamic therapy. Perhaps the most widely studied class of photosensitizers are the tetrapyrrolic macrocyclic compounds. A photosensitizer developed on porphyrin basis is Photofrin® (Axcan Pharma Inc., US) currently being used in clinical treatment. Photofrin® is an oligomer mixture of hematoporphyrins having different pharmacokinetic properties. Additionally, Photofrin® processes a non-comfortable absorption maximum which is in the range of the absorption of haemoglobin.

WO 01/74818 describes chlorines as photoselective compounds for photo-dynamic therapy. Chlorins are compounds that differ from porphyrins in that one of the pyrrole rings has been reduced, either by addition of hydrogen or by carbon bond formation. Chlorins have an increased absorption in the red region which is very attractive for PDT. But the general disadvantage of these compounds is their sensitivity to oxidation under physiological conditions. This also applies to m-tetrahydroxyphenylchlorin whose in vivo activity has been studied intensely (Blant, S. A. et. al., “In vivo fluence rate effect in photodynamic therapy of early cancers with tetra(m-hydroxyphenyl)chlorin”, Photochem. Photobiol. 64, 963-968 (1996)).

A further group of known photosensitizers are the phorbides. These compounds are tetrapyrroles derived from the naturally occurring chlorophyll and having absorptions around 670 nm. A main representative of this group is pheophorbide a whose photodynamic activity has been shown in vitro and in vivo (compare e.g. DD 248 282 A1). Phorbides have the disadvantage that their synthesis is quite difficult and requires vast resources.

Further potential photosensitizers derived from meso-tetraarylporphyrins are described from Richeter, S. et. al. in J. Org. Chem. 2003, 68, 9200-9208. The described compounds are oxonaphthoporphyrins, where one pyrrole and a neighboring aryl group are fused with a keto function, or dioxoanthroporphyrins, where two pyrrols and the aryl group in meso-position between them are fused each with a keto function. The diketones show a bathochromic shift of the UV-vis absorption, the longest wave-length bands absorbing in the 700-825 nm range.

It is an object of the present invention to provide photosensitizers for PDT treatment having excellent light absorption properties and, especially, being able to generate high singlet oxygen quantum yields under irradiation.

It is another object of the present invention to provide photosensitizers which are stable under physiological conditions, soluble in water and completely obtainable by chemical synthesis.

It is still another object of the present invention to provide an efficient method of photodynamic therapy, especially for cancer treatment.

The purpose of the present invention is achieved by providing the novel porphyrin derivates of formula I

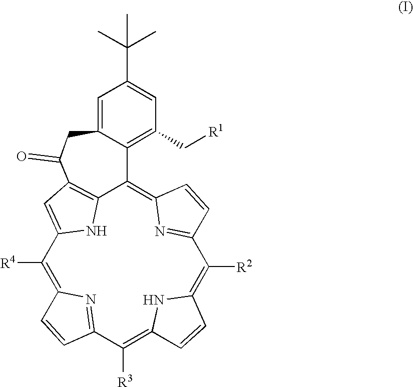

wherein
R¹ represents hydrogen, COR⁵, CO₂R⁵, CONR⁵R⁶, SO₂R⁵, SO₂NR⁵R⁶ or
R¹ represents a carboxy group whose C-atom is covalently bound to the C-atom of position 7 of the porphyrin chromophor forming a keto function;
R², R³ and R⁴ are independently selected from unsubstituted or substituted C₁-C₆ alkyl, unsubstituted or substituted aryl;
R⁵ and R⁶ are independently selected from the group comprising hydrogen, unsubstituted or substituted C₁-C₆ alkyl, unsubstituted or substituted aryl, halogen, C₁-C₅ alkoxy, alkylether, carboxylic acid or acid salts, carboxylic acid esters, amino, nitro, amino acids or their derivates, pharmaceutically acceptable salts.

The inventors of the present invention discovered that the compounds of formula I show excellent photophysical properties. Their absorption maximum is in the range of 680-700 nm, that means far from the absorption maximum of haemoglobin (613 nm). But above all, the compounds of formula I possess a high triplet quantum yield of about 95% and long triplet lifetime so that superior singlet molecular oxygen quantum yields of about 90% are provided. As it can be taken from Encyclopedia of Analytical Chemistry, R. A. Meyers (Ed.), pp. 302-322, “Photodynamic Therapy”, Table 2, John Wiley & Sons Ltd., Chichester, 2000, the singlet molecular quantum yields of porphyrins described there are only about 50 to 60%.

That means, the compounds of formula I have been found as being excellent photosensitizers for photodynamic therapy because they possess the following properties;
a) they are stable under physiological conditions,
b) they are well-soluble compounds, by adapting R¹-R⁴ the water solubility can be adjusted,
c) they are obtainable by simple chemical synthesis,
d) under irradiation they generate singlet oxygen quantum yields of about 90% ensuring a cell killing effect against unwanted tissues, especially tumors.

In a preferred embodiment the present invention relates to compounds according to formula i, wherein R¹ is hydrogen or wherein R¹ represents a carboxy group which C-atom is covalently bound to the C-atom of position 7 of the porphyrin macrocycle.

It is further preferred that R², R³ and R⁴ are independently selected from an unsubstituted or substituted aryl group, especially phenyl or phenyl which is substituted with tert-butyl. In a more preferred embodiment R², R³ and R⁴ are 4-tert-butylphenyl.

In another embodiment of the invention R², R³ and R⁴ independently from each other represent C₁-C₆ alkyl, preferably C₁-C₄ alkyl.

In a most preferred embodiment the present invention relates to compounds FB-1-CO(R¹=hydrogen, R²=R³=R⁴=4-t-butylphenyl) and FB-2-CO(R¹ is a carboxy group whose C-atom is linked with the C-atom of position 7 of the neighboring pyrrole of the porphyrin macrocycle). These special compounds are depicted in FIG. 4. It is also preferred that these compounds are encapsulated in liposomes as carrier systems to promote the transport of the compounds into target tissue.

The tetraaryl compounds of formula I have been prepared starting with the known Zn-complexes of tetraaryl porphyrin systems bearing in X²- and/or X⁶-position (X=5, 15) bromomethyl groups [“o-(Bromomethyl)Substituted Tetraaryl-porphyrin Building Blocks” N. Jux, Org. Lett 2000, 2, 2129-2132; “Introducing the Staudinger phosphazene reaction to porphyrin chemistry” Nga Hoang Huyen, Ulrike Jannsen, Hanaa Mansour, Norbert Jux, J. Forphyrins Phthalocyanines 2004). The syntheses have been carried out with derivates having up to four bromomethyl groups. The synthesis of mono, di, tri and tetraphenyl porphyrins is well known in the art (compare “The Porphyrins” Ed. D. Dolphin, Academic Press, 1978, Volume 1, chapter 3, 88-90; Gunter, M. J., Mander, L. N., J. Org. Chem. 46, 4792-4795, 1981). Such compounds can be widely functionalized as the aromatic rings may possess different substituents.

Below, as non-limiting examples for the synthesis of the compounds of formula I the preparation of FB-1-CO and FB-2-CO is described in detail. Other compounds of formula I are prepared in analogy starting with compounds having the desired substituents in the porphyrin macrocyclus. The synthesis of porphyrins with R², R³ and R⁴=alkyl is performed in a similar fashion with aliphatic aldehydes as starting materials. Typical procedures are outlined in the literature (an excellent summary can be found in K. M, Kadish, K. M. Smith, R. Guilard (Eds.): J. S. Lindsey, The Porphyrin Handbook, Vol. 1, Chapter 2: “Synthesis of meso-Substituted Porphyrins”, Academic Press, San Diego 2000, pp 45-112.

In still another preferred embodiment of the invention R¹ in formula I represents a carbonyl, carboxyl or amino group which can be covalently bound to a biologically active group, for instance dendrimer(s), lipid(s), micelle forming agent(s), antibodies or antibody fragments to promote the transport and/or the selective accumulation of the photosensitizers of formula I into target tissue. Suitable dendrimers are for instance described in S. Hackbarth et. al., Optics Communications 248 (2005), 295-306 and WO 01/08704 A2. To these dendrimers a large number of compounds of formula I can be attached forming a carrier unit to deliver the photosensitizers to the target tissue. As micelle forming agents all known tensides are applicable, for instance long chain fatty acids. Preferably used micelle forming agents are Tween 80 and Cremophor® EL. Preferred lipids for linking are also glycolipids and phospholipids, especially glycerophospholipids and LDL. Antibodies or antibody fragments which can be covalently bound to R¹ are preferably monoclonal antibodies and they are selected to have sufficient affinity to the antigen of the tissue to be treated.

Hence, porphyrin derivates of formula I which are covalently linked trough the R¹ substituent to a carrier selected from the group consisting of dendrimers, lipids, micelle forming agents, antibodies or antibody fragments, polypropylene glycol, polyethylene glycol (PEG), 2-hydroxypropyl methacrylate, polyvinylpyrrolidone (PVP) are also an object of the present invention.

Amino acids or their derivates can also be useful biologically active substituents attached to the R¹ substituent, such as for instance valine, leucine, isoleucine, threonine, methionine, phenylalanine, tryptophan, alanine, aspartic acid, cystine, cysteine, glutamic acid, glycine, histidine, proline, serine, tyrosine, asparagines, glutamine and derivates derived from them.

A further object of the present invention are porphyrin derivates of formula I encapsulated in liposomes. Used liposomes are for instance phosphatidylcholine, phosphatidylethanolamine or phosphatidylserine based liposomes. A preferred example of a phosphatidylcholine based liposome is for instance DPPC (DL-α-dipalmitoylphosphatidylcholine).

According to the invention pharmaceutically acceptable or physiologically tolerable salts of compounds of formula I are alkali or alkaline earth salts, particularly sodium, potassium, calcium or ammonium salts.

In the meaning of the invention, alkyl represents a branched or unbranched hydrocarbon chain. Thus, C₁-C₄ alkyl for instance includes methyl, ethyl, n-propyl, i-propyl, n-butyl, i-butyl, sec-butyl and tert-butyl. “Aryl” represents in the meaning of the invention substituted or unsubstituted phenyl, naphthyl or anthryl rings.

According to the invention “substituted alkyl or aryl” means substituted with alkoxy, hydroxy, halogen, amino or carboxy groups. Alkoxy represents a branched or unbranched hydrocarbon chain with one or more intervening oxygen atoms.

The present invention also relates to the compounds of formula I for use as photosensitizers in photodynamic therapy of unwanted tissue, tumor, cancer and malignant tissue. The compounds of formula I can be used for the therapeutic treatment of a broad range of tumors. Examples of such tumors are breast cancer, skin cancer, especially basal cell carcinoma, lung cancer, cancer of the urinary bladder, esophageal cancer, gastric cancer, enteric cancer, uterine cancer, ovarian cancer, pancreatic cancer, sarcomas, hepatic cancer, cancer of the upper jaw, cancer of the bile duct, cerebral tumor, malignant goiter, prostatic cancer, cancer of the parotid gland, Hodgkin's disease, multiple myeloma, renal cancer, leukaemia, and malignant lymphocytoma.

The compounds of the present invention are also useful for the treatment of ophtalmological disorders such as age-related macula degeneration, diabetic retinopathy and choroidal neovascularization; dermatological disorders such as psoriasis-urological disorders such as condyloma virus and cardiovascular disorders such as restenosis and atherosclerotic plaques. The compounds of formula I can also be used for the treatment of viral infections, especially Herpes simplex, or bacterial infections, especially infections with E. coli, Candida, Streptococcus and generally gram positive bacteria.

Therefore, the present invention also relates to a method for treating a target tissue in a mammal comprising administering to a mammal an effective amount of at least one compound of formula I which is selectively taken up and/or retained by the tissue and delivering after a certain period of time light of the appropriate wavelength to the target tissue to be absorbed by the photoselective compounds of formula i. This activating light excites the photosensitizer of formula I to generate singlet molecular oxygen destroying the tissue. The source of irradiation for this phototherapy is not limited, but a laser beam is preferable because intensive light rays in the described wavelength range can be selectively applied. Preferably the irradiation is carried out by laser light from the tip of quartz fibers. Besides the irradiation of the surface of a tumor, the internal part of the tumor can be irradiated by inserting the tip of quartz fibers into the tumor. The irradiation can be visually observed or imaged on a CRT screen.

In the photodynamic treatment the special compounds of formula I as described above are preferably used.

The present invention further concerns the use of compounds of formula I for the preparation of a pharmaceutical composition for photodynamic therapy and the pharmaceutical composition comprising as active ingredient a compound according to formula I. The pharmaceutical composition is adapted to the chosen route of administration, i.e., intravenously, orally, subcutaneously or topically. For this the pharmaceutical composition comprises besides the compound of formula I a pharmaceutically acceptable carrier. As used herein, “a pharmaceutically acceptable carrier” includes any and all solvents, dispersion media, excipients to formulate tablets, pills, capsules, creams, salve formulations, spray solutions or suspensions. In a preferred embodiment of the invention the pharmaceutical compositions for topical administration, e.g. directly to tumors, comprise liposomes, micelles, ointments, gels, hydrogels, and oils. It is well known in the art wow to formulate these pharmaceutical compositions.

The amount of active compound of formula I in such therapeutically useful compositions is such that a suitable dosage will be obtained. In a preferred embodiment the compounds of formula I are administered in a dosage of 0.1 to 5 mg per kilogram body weight/day.

### BRIEF DESCRIPTION OF THE FIGURES

FIG. 1 presents the absorption spectrum of FB-1-CO in ethanol (FB-1-CO represents the compound of formula I with R¹=hydrogen and R²=R³=R⁴=4-t-butylphenyl). The band at 690 nm represents the first exited singlet state of FB-1-CO.

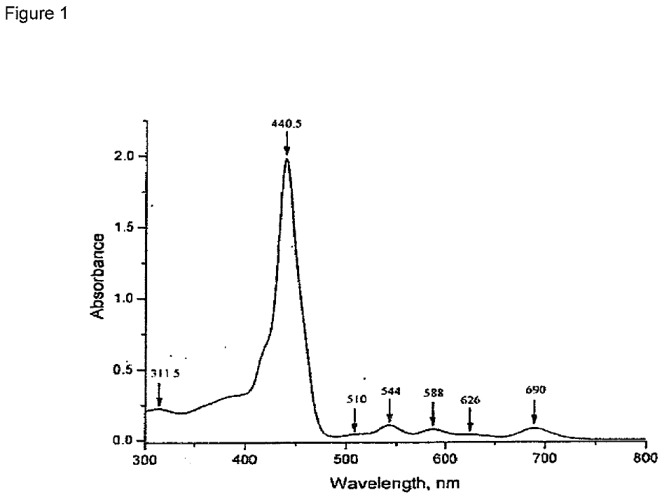

FIG. 2 presents the fluorescence spectrum of FB-1-CO. The fluorescence quantum yield has been determined to 0.02.

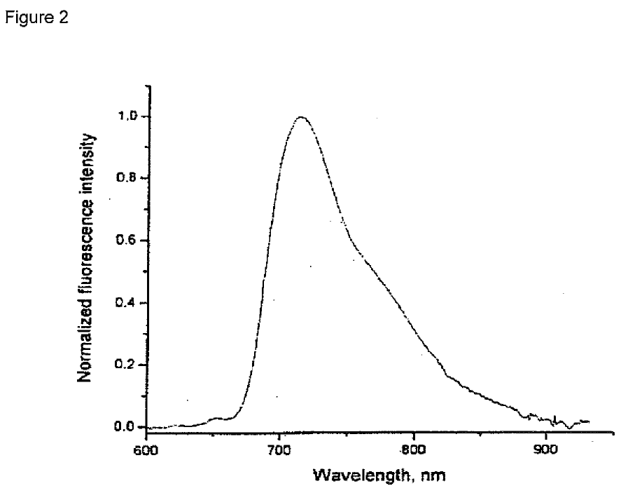

FIG. 3 presents the preferred compounds of formula I of the present invention.

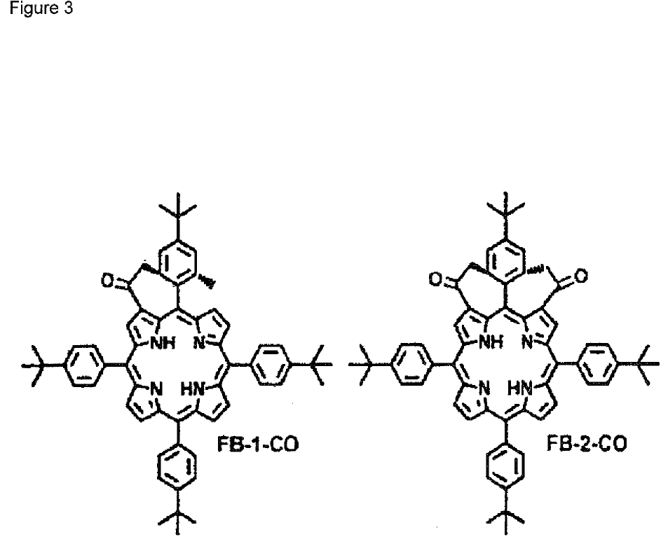

FIG. 4 outlines the chemistry involved in the synthesis of FB-1-CO.

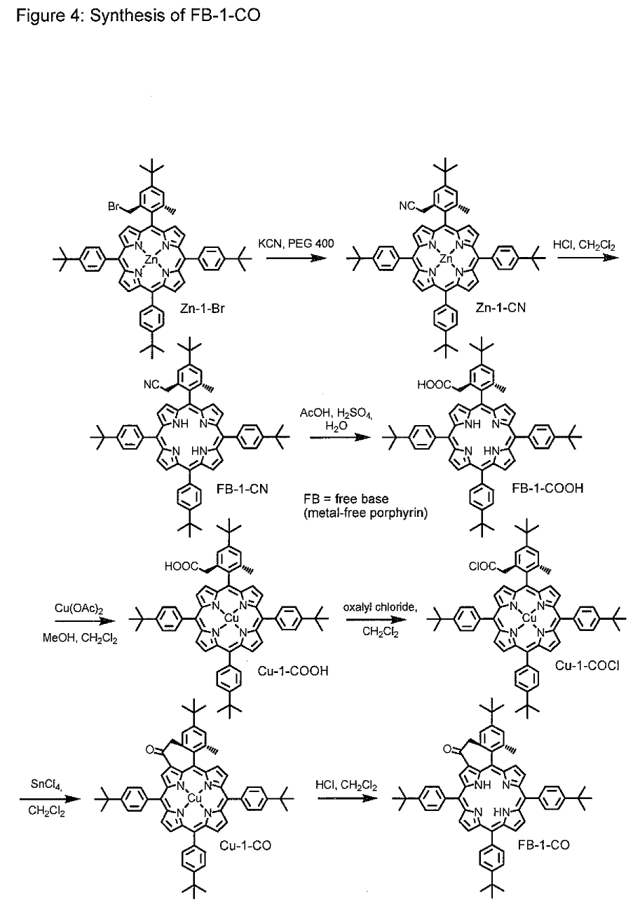

FIG. 5 outlines the chemistry involved in the synthesis of FB-2-CO.

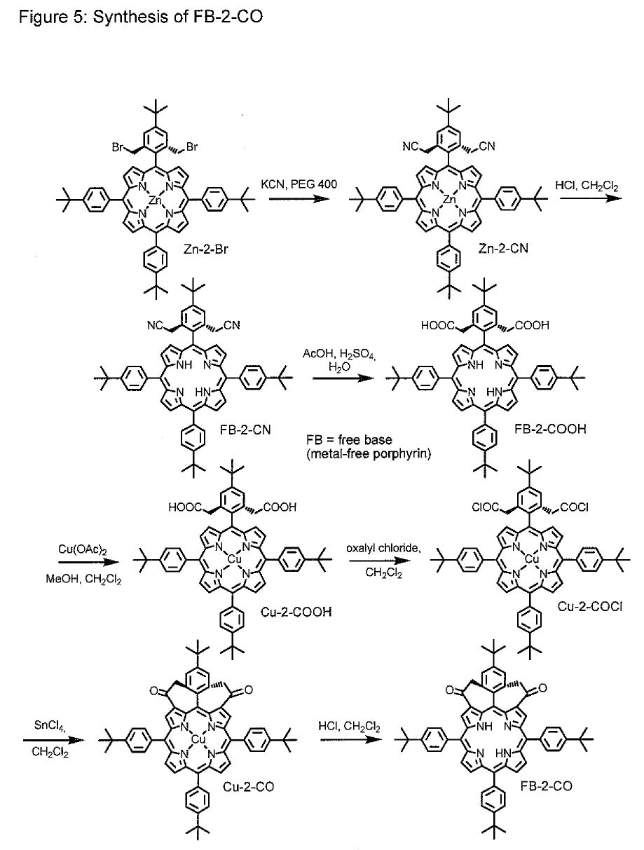

FIG. 6 outlines the phototoxicity of FB-1-CO (FBM). Cells were incubated with FB-1-CO delivered in liposome preparations (FBM/Lip 10 μM and FBM/Lip 37.5) or dissolved in DMSO (FBM/DMSO 2 μM and FBM/DMSO 5 μM). Control cells were incubated with blank liposomes (Lip). After 24 h incubations cells were irradiated with a LED (LED 700, max. em. at 700 nm) and with a UVA lamp (UVA-400/96w and UVA-400/24w, max em. at 400 nm) at distances of 1 and 2 cm. Cell vitality and proliferation percentages were determined with respect to reference cells (R) which were only treated with light.

The following examples are given to highlight a preferred mode of synthesizing compounds of formula I, determining their photophysical properties and their phototoxity in vitro.

### EXAMPLES

The preparation of this class of compounds is based on the o-(bromomethyl)-substituted tetraarylporphyrin building blocks developed by Jux [“o-(Bromomethyl)Substituted Tetraaryl-porphyrin Building Blocks” Norbert Jux, Org. Lett. 2000, 2, 2129-2132; “Introducing the Staudinger phosphazene reaction to porphyrin chemistry” Nga Hoang Huyen, Ulrike Jannsen, Hanaa Mansour, Norbert Jux, J. Porphyrins Phthalocyanines 2004, 8, 1356-1365]. The following reaction pathway is illustrated for the preparation of compounds FB-1-CO and FB-2-CO, exemplarily. Other compounds of this class have been synthesized using the same protocols with minor changes concerning amounts of solvents or reaction times. The chemical transformations are based on well known standard methods.

Column chromatography was performed on silica gel 32-63 μm, 60 Å, MP Biomedicals. ¹H and ¹³C NMR spectra were recorded on JEOL JMM EX 400, and JEOL GX 400 instruments. FAB mass spectrometry was performed with Micromass Zabspec and Varian MAT 311A machines. Standard UV/VIS spectra were recorded on a Shimadzu UV-3102 PC UV/Vis NIR scanning spectrophotometer. IR spectra were either taken with a Bruker Vector 22 spectrometer or an ASI React IR-1000 spectrometer. Elemental analysis were done with EA 1110 CHNS from CE Instruments. Analytical thin layer chromatography (TLC) was performed on Merck 60 F254 silica gel (precoated on aluminium) with a layer thickness of 0.2 mm.

### Example 1

### Preparation of 5⁴, 10⁴, 15⁴, 20⁴-tetra-t-butyl[3,5²]-ethano-5⁶-methyl-3¹-oxo-5,10,15 20-tetraphenylporphyrin (FB-1-CO)

### Brief Summary of the Synthesis of FB-1-CO:

The mono-bromomethyl porphyrin Zn-1-Br (zinc 5²-bromomethyl-5⁴, 10⁴, 15⁴, 20⁴-tetra-t-butyl-5⁶-methyl-5,10,15,20-tetraphenylporphyrin) was reacted with potassium cyanide in PEG400 to give the monocyano porphyrin Zn-1-CN (zinc 5⁴, 10⁴, 15⁴, 20⁴-tetra-t-butyl-5²-(cyanomethyl)-5⁶-methyl-5,10,15,20-tetraphenylporphyrin). Demetallation (FB-1-CN) and acidic saponification of the nitrile substituent gave the monocarboxy porphyrin FB-1-COOH (5⁴, 10⁴, 15⁴, 20⁴-tetra-t-butyl-5⁶-methyl-5,10,15,20-tetraphenylporphyrin-5²-ethanoic acid) which after metallation with cupper(II) chloride gave the copper porphyrin Cu-1-COOH (copper(II) 5⁴, 10⁴, 15⁴, 20⁴-tetra-t-butyl-5⁶-methyl-5,10,15,20-tetraphenylporphyrin-5²-ethanoic acid). The acid was transformed into the acid chloride (not isolated) with oxalyl chloride and reacted with tin(IV) chloride to give the copper complex Cu-1-CO (copper(II) 5⁴, 10⁴, 15⁴, 20⁴-tetra-t-butyl[3,5²]-ethano-5⁶-methyl-3¹-oxo-5,10,15,20-tetraphenylporphyrin). Demetallation with acid gave the desired free-base system FB-1-CO (5⁴, 10⁴, 15⁴, 20⁴-tetra-t-butyl[3,5²]-ethano-5⁶-methyl-3¹-oxo-5,10,15,20-tetraphenylporphyrin.

### Protocol:

1. 200 mg (211 μmol) of FB-1-Br (5²-bromomethyl-5⁴, 10⁴, 15⁴, 20⁴-tetra-t-butyl-5⁶-methyl-5,10,15,20-tetraphenylporphyrin) were dissolved in 30 ml of dichloromethane. To this solution a saturated solution of 0.93 g (4.22 mmol) zinc acetate (Zn(O₂CCH₃)₂×2 H₂O) in methanol was added and the reaction mixture was stirred at room temperature. When TLC control indicated completion, water was added and the layers were separated. After washing the organic layer three times with water, the residing solution was dried over magnesium sulfate and evaporated to dryness to give Zn-1-Br (zinc 5² bromomethyl-5⁴, 10⁴, 15⁴, 20⁴-tetra-t-butyl-5⁶-methyl-5,10,15,20-tetraphenylporphyrin).

2. To Zn-1-Br (zinc 5²-bromomethyl-5⁴, 10⁴, 15⁴, 20⁴-tetra-t-butyl-5⁶-methyl-5,10,15,20-tetraphenylporphyrin), which was obtained by step 1, 546 mg (8.93 mmol) of potassium cyanide and 25 ml polyethylene glycol 400 (PEG 400) were added and the reaction mixture was stirred at room temperature for 24 hours. Afterwards water and dichloromethane were added, the layers separated and the organic layer was finally washed several times with water in order to guarantee absence of free cyanide ions. After evaporation of the solvent, Zn-1-CN (zinc 5⁴, 10⁴, 15⁴, 20⁴-tetra-t-butyl-5²-(cyanomethyl)-5⁶-methyl-5,10,15,20-tetraphenylporphyrin) was obtained as purple crystals.

3. To Zn-1-CN (zinc 5⁴, 10⁴, 15⁴, 20⁴-tetra-t-butyl-5²-(cyanomethyl)-5⁶-methyl-5,10,15,20-tetraphenylporphyrin), which was obtained by step 2, half-concentrated aqueous hydrochloric acid was added and the biphasic mixture was vigorously shaken for five minutes. The organic layer was then separated and washed with water, aqueous sodium bicarbonate solution and water. After drying over magnesium sulfate the solution was evaporated to dryness to yield FB-1-CN as a deep violet solid. (Yield: 178 mg, 199 μmol, 94% after steps 1-3).

4. FB-1-CN (54, 104, 154, 20⁴-tetra-t-butyl-5²-(cyanomethyl)-5⁶-methyl-5,10,15,20-tetraphenylporphyrin), which was obtained by step 3, was dissolved in 15 ml of glacial acetic acid. Then 15 ml concentrated sulfuric acid and 5 ml of water were added and the reaction mixture was stirred at 90° C. for 80 hours. Afterwards the mixture was poured on crushed ice and left standing until FB-1-COOH (5⁴, 10⁴, 15⁴, 20⁴-tetra-t-butyl-5⁶-methyl-5,10,15,20-tetraphenylporphyrin-5²-ethanoic acid) precipitated completely. The precipitate was filtered of and taken up in dichloromethane. This solution was washed several times with water, dried over magnesium sulfate and finally concentrated to a volume of about 50 ml.

5. To this solution of FB-1-COOH (5⁴, 10⁴, 15⁴, 20⁴-tetra-t-butyl-5⁶-methyl-5,10,15,20-tetraphenylporphyrin-5²-ethanoic acid), which was obtained by step 4, a solution of 687 mg (3.78 mmol) copper acetate (Cu(O₂CCH₃)₂) in 30 ml of methanol and some drops of glacial acetic acid were added and the reaction mixture was stirred for 15 hours at room temperature. Afterwards the reaction mixture was evaporated to dryness and the residue was taken up in dimethyl formamide. The solution was then poured onto a mixture of crushed ice and glacial acetic acid whereupon the copper complex precipitated. After filtration the residue was washed with water, taken up in dichloromethane, the solution dried over magnesium sulfate and finally evaporated to dryness yielding Cu-1-COOH (copper(II) 5⁴, 10⁴, 15⁴, 20⁴-tetra-t-butyl-5⁶-methyl-5,10,15,20-tetraphenylporphyrin-5²-ethanoic acid) as a bright red powder.

6. Cu-1-COOH (copper(II) 5⁴, 10⁴, 15⁴, 20⁴-tetra-t-butyl-5⁶-methyl-5,10,15,20-tetraphenylporphyrin-5²-ethanoic acid), which was obtained by step 5, was taken up in 50 ml of dry dichloromethane under an inert atmosphere and an excess of oxalyl chloride (1 ml) was added. This solution was stirred at room temperature for 90 minutes and finally evaporated to dryness at elevated temperature. The brownish residue was dissolved in 50 ml of dry dichloromethane under an inert atmosphere and an excess of tin(IV) chloride (0.5 ml) was added. After stirring at room temperature for ten minutes, the reaction was quenched by slowly adding a saturated aqueous sodium bicarbonate solution. Extraction with dichloromethane furnished a green solution which was washed with water, dried over magnesium sulfate and evaporated to dryness to give Cu-1-CO (copper(II) 5⁴, 10⁴, 15⁴, 20⁴-tetra-t-butyl[3,5²]-ethano-5⁶-methyl-3¹-oxo-5,10,15,20-tetraphenylporphyrin).

7. Cu-1-CO (copper(II) 5⁴, 10⁴, 15⁴, 20⁴-tetra-t-butyl[3,5²]-ethano-5⁶-methyl-3¹-oxo-5,10,15,20-tetraphenylporphyrin), which was obtained by step 6, was taken up in 10 ml of trifluoroacetic acid and 2 ml of concentrated sulfuric acid. The reaction mixture was stirred at room temperature for 30 minutes. After addition of water and dichloromethane the layers were separated, the organic layer washed with water, aqueous sodium bicarbonate solution and water. After drying over magnesium sulfate the solution was evaporated to dryness to yield a green-violet solid. This was chromatographed over silica using dichloromethane and hexanes (ratio=1:2) as eluent giving FB-1-CO (5⁴, 10⁴, 15⁴, 20⁴-tetra-t-butyl[3,5²]-ethano-5⁶-methyl-3¹-oxo-5,10,15,20-tetraphenylporphyrin) as a dark green powder. (yield: 107 mg, 120 μmol, 57% after steps 1-7).

Characterization data for FB-1-CO:

¹H NMR (400 MHz, CDCl₃, rt), δ [ppm]=8.96 (s, 1H), 8.89 (d, 1H, ³J=4.9 Hz), 8.79 (m, 2H), 8.74, 8.67, 8.63 (3d, 1H each, ³J=4.9 Hz), 8.32-7.69 (div. m's, 12H), 7.68 (d, 1H, ⁴J=1.7 Hz), 7.35 (d, 1H, ⁴J=1.7 Hz), 5.49 (d, 1H, ²J=11.7 Hz), 4.11 (d, 1H, ²J=11.7 Hz), 1.59 (s, 9H), 1.58 (s, 18H), 1.50 (s, 9H), 1.17 (s, 3H), −1.62 (s, 2H).

¹³C NMR (100.5 MHz, CDCl₃, rt), δ [ppm]=192.4, 152.3, 151.2, 150.9, 150.8, 142.1, 138.6, 138.5, 138.1, 137.7, 136.1, 134.7, 134.3, 128.3, 126.1, 125.8, 124.6, 124.2, 124.0, 123.9, 123.8, 122.0, 119.0, 114.0, 53.7, 34.9, 34.8, 34.7, 31.6, 31.4, 22.5.

MS (FAB, NBA), m/z=893.

UV/Vis (CH₂Cl₂), λ [nm] (ε [I·mol⁻¹·cm⁻¹])=442 (223000), 543 (11300), 587 (9000), 626 (4100), 689 (9000).

IR (KBr), \tilde{ν} [cm⁻¹]=3316, 3029, 2957, 2903, 2868, 1683, 1606, 1552, 1498, 1475, 1393, 1363, 1351, 1262, 1239, 1197, 1154, 1108, 1019, 984, 965, 869, 850, 799, 718.

| | | | | |
|---|---|---|---|---|
| EA: C₆₃H₆₄N₄O × 2H₂O: | calculated | C 81.43 | H 7.38 | N 6.03 |
| | found | C 81.26 | H 7.54 | N 5.74 |

### Example 2

### Preparation of 5⁴, 10⁴, 15⁴, 20⁴-tetra-t-butyl-[3,5²], [5⁶,9]-bis-ethano-3¹,9¹-dioxo-5,10,15,20-tetraphenylporphyrin (FB-2-CO)

### Brief Summary of the Synthesis of FB-1-CO:

The bis-bromomethyl porphyrin Zn-2-Br (zinc 5²,5⁶-bis-(bromomethyl)-5⁴, 104, 154, 204-tetra-t-butyl-5,10,15,20-tetraphenylporphyrin) was reacted with potassium cyanide in PEG400 to give the dicyano porphyrin Zn-2-CN (zinc 5⁴, 10⁴, 15⁴, 20⁴-tetra-t-butyl-5²,5⁶-bis-(cyanomethyl)-5,10,15,20-tetraphenyporphyrin). Demetallation (FB-2-CN) and acidic saponification of the nitrile substituents gave the dicarboxy porphyrin FB-2-COOH (5⁴, 10⁴, 15⁴, 20⁴-tetra-t-butyl-5,10,15,20-tetraphenylporphyrin-5²,5⁶-bis-ethanoic acid) which after metallation with cupper(II) chloride gave the copper porphyrin Cu-2-COOH (copper(II) 5⁴, 10⁴, 15⁴, 20⁴-tetra-t-butyl-5,10,15,20-tetraphenylporphyrin-5²,5⁶-bis-ethanoic acid). The acid was transformed into the acid chloride (not isolated) with oxalyl chloride and reacted with tin(IV) chloride to give the copper complex Cu-2-CO (copper(II) 5⁴, 10⁴, 15⁴, 20⁴-tetra-t-butyl-[3,5²],[5⁶,9]-bis-ethano-3¹,9¹-dioxo-5,10,15,20-tetraphenylporphyrin). Demetallation with acid gave the desired free-base system FB-2-CO (5⁴, 10⁴, 15⁴, 20⁴-tetra-t-butyl-[3,5²],[5⁶,9]-bis-ethano-3¹,9¹-dioxo-5,10,15,20-tetraphenylporphyrin).

### Protocol

The synthesis of FB-2-CO was exactly performed in the same way as the generation of FB-1-CO. Reaction conditions and yields are comparable.

Characterization data for FB-2-CO:

¹H NMR (400 MHz, CDCl₃, rt), δ [ppm]=8.69 (s, 2H), 8.64 (d, 2H, ³J=4.9 Hz), 8.54 (d, 2H, ³J=4.9 Hz), 8.06 (d, 6H, ³J=7.9 Hz), 7.76 (d, 6H, ³J=7.9 Hz), 7.70 (s, 2H), 4.37 (d, 2H, ²J=11.3 Hz), 3.84 (d, 2H, ²J=11.3 Hz), 1.58 (s, 18H), 1.58 (s, 9H), 1.51 (s, 9H), −1.33 (s, 2H).

¹³C NMR (100.5 MHz, CDCl₃, rt), δ [ppm]=190.0, 153.8, 151.6, 151.3, 150.8, 137.7, 137.5, 136.0, 134.7, 128.9, 127.8, 125.6, 124.3, 124.2, 122.9, 111.8, 53.9, 35.0, 34.9, 31.6, 31.3.

MS (FAB, NBA): m/z=920.

UV/Vis (CH₂Cl₂), λ [nm] (ε [I·mol⁻¹·cm⁻¹])=349 (28500), 462 (103700), 486 (70200), 597 (6500), 627 (12000), 733 (11200).

IR (KBr), \tilde{ν} [cm⁻¹]=3034, 2961, 2907, 2868, 1695, 1552, 1502, 1459, 1393, 1363, 1247, 1197, 1135, 1108, 1027, 1000, 980, 965, 869, 849, 799, 714.

| | | | | |
|---|---|---|---|---|
| EA: C₆₄H₆₄N₄O₂ × 2H₂O: | calculated | C 80.47 | H 6.96 | N 5.87 |
| | found | C 80.28 | H 6.93 | N 5.70 |

### Example 3

### Determination of the Photophysical Properties of compound FB-1-CO Prepared in Example 1

The ground state absorption spectra were recorded at room temperature using a commercial spectrophotometer Shimadzu UV-2501PC. Steady-state fluorescence spectra were measured in 1 cm×1 cm quartz optical cells using a combination of a cw-Xenon lamp (XBO 150) and a monochromator (Lot-Oriel, bandwidth 10 nm) for excitation and a polychromator with a cooled CCD matrix as a detector system (Lot-Oriel, Instaspec IV).

Photosensitizer-generated time-resolved singlet oxygen luminescence was measured at 1270 nm. A nanosecond Nd-YAG laser (BMI) equipped with an OPO (BMI) was used to excite the samples at 510 nm and the luminescence signal was recorded by a germanium pin diode (Northcoast). To calculate the singlet oxygen quantum yield, (DA, the solution of Pheo in DMF as well as in ethanol were used as reference (Φ_{Δ}^{Pheo}=0.52).

To measure transient absorption spectra (TAS), a white light continuum was generated as a test beam in a cell with D₂O/H₂O mixture using intense 25 ps single pulses from a Nd:YAG laser (Ekspla) at 1064 nm. Before passing through the sample, the continuum radiation was split to get a reference spectrum. The transmitted as well as the reference beam were focused into two optical fibers and were recorded simultaneously at different traces on a CCD-matrix (Lot-Oriel, Instaspec IV). The tunable radiation of a OPO/OPG (Ekspla, tuning range 210-2300 nm) pumped by the third harmonic of the same laser was used for excitation of the samples. The mechanical delay line allowed measuring the light-induced changes in the absorption spectrum at different time delays up to 15 ns after excitation.

Thus, the following photophysical properties have been determined:

Q_{y}−absorption at λ=690 nm
fluorescence quantum yield: Φ_{FI}=0.02
singlet oxygen quantum yield: Φ_{Δ}=0.9
ISC quantum yield: Φ_{ISC}=0.95±0.05

### Example 4

### Demonstration of the Phototoxity of Compound FB-1-CO Prepared in Example 1

### Liposomes Preparation

DL-α-dipalmitoylphosphatidyicholine (C16:0) (DPPC, Sigma, Germany) liposomes were prepared using the injection method [Kremer et al. 1992]. DPPC (˜4.2 mg) was dissolved in ethanol. A DMSO solution of FB-1-CO (1×10⁻³ M) was added to the DPPC solution. The mixture was injected into 2 ml of Dulbecco's phosphate buffered saline (PBS, Sigma, Germany) using a 100 μL Hamilton syringe. PBS was quickly stirred and at the temperature of 60° C. The resulting liposomes preparation was then sonicated (˜50W, 1 min) using a microtip probe sonicator (Merk, Germany). Three liposomal mixtures were prepared with a final FB-1-CO concentration of 0, 10 and 37.5 μM.

### Cell Culture and Incubation Conditions

A special line of human T-lymphocytes (Jurkat cells: clone E 6-1, human acute T-cell leukaemia, ACACC catalogue) was cultivated in 50 ml flasks in 5 ml RPMI 1640 medium containing Glutamax-1, supplemented with 10% foetal calf serum (FCS), 100 μg/ml streptomycin and 100 I.E./ml penicillin. Cells were cultivated at 37° C. in 100% humidity and 5% CO₂ and were seeded in new medium every 2-3 days.

Cells (2×10⁵ cells/ml) were incubated in growing medium with the studied compound delivered as a solution in DMSO (0.5%) or liposome preparation (10%). Cells incubated in growing medium with 0.5% of the organic solvent or 10% of empty liposomes were used as controls. Incubation concentrations of 10 and 37.5 μM FB-1-CO were used in medium/liposomes preparation while incubation concentration of 2 and 5 μM FB-1-CO were used in medium/DMSO preparations. After 24 h incubation time, cells were washed twice to remove that amount of the PS-compound, which had not been taken-up. Cells were then re-suspended in PBS and used in uptake of phototoxicity experiments.

### Irradiation Conditions

Cells were irradiated both with laser light at a wavelength of 700 nm and a UVA lamp. For the irradiation with laser light, a LED with an emission of 700 nm was used. Cells were placed in a 96-wells culture plate, 100 μl per well. The 96-well culture plate was positioned on a plane having a circular hole with a surface of 0.32 cm², which correspond to the surface of a single well. The laser diode is fixed on the bottom of the plane in correspondence to the wall. Cells were irradiated for a time of 1 min.

The UVA irradiation of cell suspensions was done with a fluorescence lamp (type TLK 40 W, Philips). Cells were placed in a 24-well plate and covered with a quartz glass. The fluorescence lamp has a 99.58% emission in the UVA region (315 and 400 nm) and a 0.42% emission in the spectral range below 315 nm. The culture plate was placed under the lamp at 1 and 2 cm distance and cells were irradiated for 10 min.

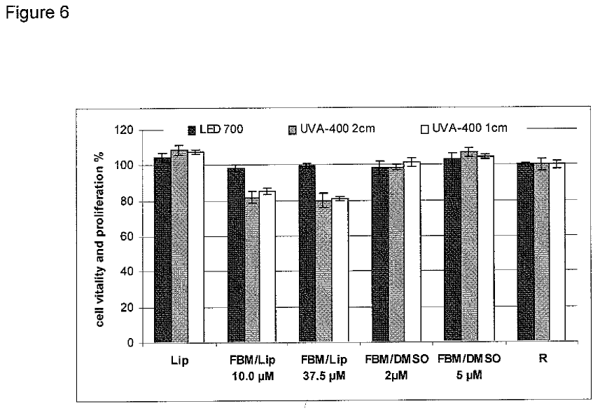

Cell vitality and proliferation was detected using the XTT test (Roche, Germany) 24 h after irradiation. In FIG. 6 the results are demonstrated. FBM in FIG. 6 means FB-1-CO.

1. A compound of formula I 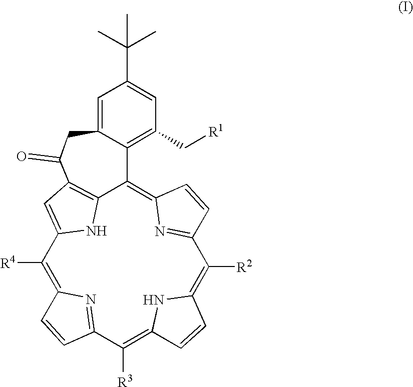
wherein
R¹ represents hydrogen, COR⁵, CO₂R⁵, CONR⁵R⁶, SO₂R⁵, SO₂NR⁵R⁶ or
R¹ represents a carboxy group which C-atom is covalently bound to the C-atom of position 7 of the porphyrin chromophor
R², R³ and R⁴ are independently selected from unsubstituted or substituted C₁-C₆ alkyl, unsubstituted or substituted aryl groups
R⁵ and R⁶ are independently selected from the group consisting of hydrogen, unsubstituted or substituted C₁-C₆ alkyl, unsubstituted or substituted aryl, halogen, C₁-C₅ alkoxy, alkylether, carboxylic acid or acid
salts, carboxylic acid esters, amino, nitro, amino acids or their derivates, a pharmaceutically acceptable salt.

2. The compound according to claim 1, wherein R¹ represents hydrogen.

3. The compound according to claim 1, wherein R¹ represents a carboxy group which C-atom is covalently bound to the C-atom of position 7 of the porphyrin macrocycle.

4. The compound according to claim 1, wherein R², R³ and R⁴ are independently selected from an unsubstituted or substituted aryl ring.

5. The compound according to claim 4, wherein the aryl group is phenyl.

6. The compound according to claim 5, wherein phenyl is substituted with tert-butyl, preferably in 4-position.

7. The compound according to claim 1, wherein R², R³ and R⁴ are independently selected from C₁-C₄ alkyl.

8. The compound according to claim 1 covalently linked to a carrier selected from the group consisting of dendrimers, lipids, micelle forming agents, antibodies or antibody fragments.

9. The compound according to claim 1 encapsulated in liposomes.

10. A compound according to claim 1 for use as photosensitizer in photodynamic therapy.

11. Use of a compound according to claim 1 for the preparation of a pharmaceutical composition for photodynamic therapy.

12. Use according to claim 11 for photodynamic therapy of tumors, dermatological disorders, viral or bacterial infections, opthalmological disorders, cardiovascular disorders or urological disorders.

13. A pharmaceutical composition comprising as active ingredient a compound according to claim 1.

14. A method of treating a tumor, dermatological disorder, viral or bacterial infection, opthalmological disorder, cardiovascular disorder or urological disorder in a patient in need of such treatment, comprising administering to said patient an effective amount of a compound of claim 1.
